(19) United States
(12) Patent Application Publication
(10) Pub. No.: US 2011/0231953 A1
(43) Pub. Date: Sep. 22, 2011

(54) TRANSFORMED PLANT WITH INCREASED CHLOROPLASTS PER CELL

(76) Inventors: Kumiko OKAZAKI, Saitama (JP); Shin-ya Miyagishima, Saitama (JP); Yukihiro Kabeya, Saitama (JP); Hiromitsu Nakanishi, Saitama (JP)

(21) Appl. No.: 12/724,725

(22) Filed: Mar. 16, 2010

(51) Int. Cl. A01H 1/00; A01H 5/00; C12N 5/04

(52) U.S. Cl. 800/278; 800/298; 435/419

(57) ABSTRACT

This invention provides a transformed plant with increased number of chloroplasts per cell, comprising an over-expressed foreign DNA which codes for PDV1 and/or PDV2, or CRF2, a homologue thereof, or a mutant thereof.

### FIELD OF THE INVENTION

This invention relates to a transformed plant with the increased number of chloroplasts per cell and the method for producing the plant.

### BACKGROUND

Chloroplasts originally derived from a bacterium related to extant cyanobacteria which was engulfed by a primary nonphotosynthetic eukaryotic host cell more than a billion years ago. Over time, the engulfed bacterial endosymbionts have been reduced to chloroplasts and vertically transmitted to subsequent generations (Reyes-Prieto et al., 2007; Gould et al., 2008). Reminiscent of their free-living ancestor, chloroplasts multiply by division. (Possingham and Lawrence, 1983; Boffey and Lloyd, 1988; Kuroiwa et al., 1998). However, most of the genes once present in the engulfed bacterial endosymbiont have been lost or transferred to the host nuclear genome; those still used by the chloroplasts are translated by the host and targeted back into the chloroplasts to express their functions. Therefore, chloroplasts cannot divide by themselves, and the division is performed by nucleus-encoded proteins.

Chloroplast division is performed by ring structures at the division site, spanning both the inside and the outside of the two chloroplast envelope membranes (Yoshida et al., 2006; Maple and Moller, 2007; Yang et al., 2008). The ring structures were identified by earlier electron microscopic studies (Kuroiwa et al., 1998; Miyagishima et al., 2001; Yoshida et al., 2006), and recent studies have identified several proteins that form a complex at the division site. Consistent with the endosymbiotic origin of chloroplasts, the division complex includes FtsZ, a self-assembling tubulin-like GTPase (Osteryoung and Vierling, 1995; Osteryoung et al., 1998; Vitha et al., 2001; Mori et al., 2001; Kuroiwa et al., 2002), and ARC6 (Accumulation and Replication of Chloroplasts 6), a J-domain containing protein, both of which are descended from the cell division machinery of the engulfed cyanobacterium (Vitha et al., 2003). In addition, the division complex includes DRP5B (Dynamin-Related Protein 5B) (also known as ARCS), a member of dynamin family of self-assembling GTPase proteins (Gao et al., 2003; Miyagishima et al., 2003), and PDV1 (Plastid DiVision 1) and PDV2 proteins. DRP5B is specific to plants and algae, and is suggested to have evolved from a dynamin-related protein that is involved in eukaryotic cytokinesis (Miyagishima et al., 2008). PDV1 and PDV2 are specific to land plants (Miyagishima et al., 2006; Glynn et al. 2008).

Chloroplast division in land plants is initiated by stromal FtsZ ring formation at the division site (containing FtsZ1 and FtsZ2, which arose by genetic duplication after the cyanobacterial endosymbiosis) (Vitha et al., 2001; Kuroiwa et al., 2002), which is stabilized by the inner envelope spanning protein ARC6 (Vitha et al., 2003). Then PDV1 and PDV2 are recruited to the division site through direct interaction between PDV2 and ARC6 (Glynn et al., 2008). In addition, a recent study showed that the recruitment of PDV1 is mediated by PARC6 (paralog of ARC6 unique to vascular plants; Glynn et al., 2009). Finally, the dynamin-related protein DRP5B is recruited by PDV1 and PDV2 (Miyagishima et al., 2006; Glynn et al., 2008), and the entire division complex is involved in the fission of the chloroplast at the division site (FIG. 1).

In algae and meristematic cells in land plants, chloroplast (or plastid) division keeps pace with cell division to ensure their passage to daughter cells during cell division. In contrast, cells of land plants regulate the chloroplast division rate in accord with cell differentiation, thereby controlling the number of chloroplasts (Possingham and Lawrence, 1983; Boffey and Lloyd, 1988). For example, small developing chloroplasts in young emerged leaves actively divide while the division rate slows down as leaves and chloroplasts get matured (Boffey and Lloyd, 1988).

Although several components that drive chloroplast division have been characterized, little is known about how the division machinery is controlled so as to modulate the rate of division in land plants.

### SUMMARY OF THE INVENTION

In one aspect, the present invention relates to a transformed plant with increased number of chloroplasts per cell, comprising an over-expressed foreign DNA which codes for a land-plant specific component protein of a division apparatus PDV1 and/or PDV2, a homologue thereof, or a mutant thereof.

In one embodiment of this aspect, the PDV1 comprises an amino acid sequence as shown in SEQ ID NO: 1, PDV1 homologue comprises an amino acid sequence having at least 20% identity to the amino acid sequence of SEQ ID NO: 1, and PDV1 mutant comprises an amino acid sequence of SEQ ID NO: 1 having deletion, addition and/or substitution of one or more amino acids and an activity of increasing the number of chloroplasts per cell when compared with wild type.

In another embodiment of this aspect, the PDV2 comprises an amino acid sequence as shown in SEQ ID NO: 2, PDV2 homologue comprises an amino acid sequence having at least 20% identity to the amino acid sequence of SEQ ID NO: 2., and PDV2 mutant comprises an amino acid sequence of SEQ ID NO: 2 having deletion, addition and/or substitution of one or more amino acids and an activity of increasing the number of chloroplasts per cell when compared with wild type.

In a further embodiment of this aspect, the DNA comprises: the DNA comprises: (a) nucleotide sequence as shown in SEQ ID NO: 4 or 5, (b) a nucleotide sequence having an at least 20% identity to the nucleotide sequence of SEQ ID NO: 4 or 5, (c) a nucleotide sequence encoding the PDV1 or PDV2 as defined in above embodiments, or (d) a nucleotide sequence capable of hybridizing with the nucleotide sequence complement to the nucleotide sequence of SEQ ID NO: 4 or 5 under strength conditions, wherein the nucleotide sequence (a), (b) or (c) codes for a protein having an activity of increasing the number of chloroplasts per cell when compared with wild type.

In other aspect, the present invention relates to a transformed plant with increased number of chloroplasts per cell, comprising an over-expressed foreign DNA which codes for a transcription factor CRF2, a homologue thereof, or a mutant thereof.

In one embodiment of this aspect, the CRF2 comprises an amino acid sequence as shown in SEQ ID NO: 3, CRF2 homologue comprises an amino acid sequence having at least 20% identity to the amino acid sequence of SEQ ID NO: 3, and CRF2 mutant comprises an amino acid sequence of SEQ ID NO: 3 having deletion, addition and/or substitution of one or more amino acids and an activity of increasing the number of chloroplasts per cell when compared with wild type.

In another embodiment of this aspect, the DNA comprises: (a) a nucleotide sequence as shown in SEQ ID NO: 6, (b) a nucleotide sequence having an at least 20% identity to the nucleotide sequence as shown in SEQ ID NO: 6, (c) a nucleotide sequence encoding CRF2 as defined above embodiment, (d) a nucleotide sequence capable of hybridizing with the nucleotide sequence complement to the nucleotide sequence of SEQ ID NO: 6 under strength conditions, wherein the nucleotide sequence (a), (b) or (c) codes for a protein having an activity of increasing the number of chloroplasts per cell when compared with wild type.

In a more aspect, the present invention relates to progeny of the transformed plant of aforementioned aspects.

In a further aspect, the present invention relates to a cell, tissue, organ, seed derived from the transformed plant or progeny thereof in aforementioned aspects.

In a still further aspect the present invention relates to a method for producing a transformed plant of any one of aforementioned aspects, comprising the following steps of: (1) introducing a vector comprising the DNA as defined in aforementioned aspects into cells of plant, (2) selecting a transformed cell over-expressing the DNA, from the transformed cells of step (1), and (3) generating the transformed plant from the transformed cells of step (2)

### BRIEF DESCRIPTION OF THE DRAWINGS

FIG. 1. Diagram showing the pathway of chloroplast division complex assembly (Yang et al., 2008). Only the known division-site-localized components are shown.

FIG. 2. Over-expression of PDV1 and PDV2 increases the number of chloroplasts. Chloroplasts of wild type (WT), transgenic plants over-expressing PDV1, PDV2, and both PDV1 and PDV2 (35S-PDV1, 35S-PDV2 and 35S-PDV1 35S-PDV2) and pdv2/PDV2 and pdv2/pdv2 T-DNA insertional mutants. Tips of the first true leaves were cut from 3-week-old or less plants grown on agar plates. Single leaf mesophyll cells observed by Nomarski optics are shown. There are no visible differences in growth among these lines. Bar=20 μm.

FIG. 3. Statistical comparison of the number of chloroplasts per mesophyll cell. Error bars represent standard deviation (n=50 cells).

FIG. 4. Immunoblot and RT-PCR analyses showing the levels of PDV2 protein and the PDV1 transcript. The same amount of total protein extracted from rosettes was analyzed by anti-PDV2 antibodies. The Rubisco large subunit stained by Ponceau S is shown as the loading control. Three biological replicates showed the same result for the immunoblot analyses. Total RNA extracted from russets was used for RT-PCR to examine the PDV1 transcript level. UBQ1 was used as the internal control. The number of PCR cycles was 28 for PDV1 and 26 for UBQ1. The signals were estimated by ethidium bromide staining. Two biological replicates showed the same result.

FIG. 5. Change of chloroplast size during leaf development. Chloroplasts in a young emerging leaf (1) and expanding leaves (2-4) of the wild-type plant were observed by Nomarski optics. Chloroplasts are observably still dividing in the first true leaf (4, indicated by arrow). Bars=2 mm (left) and 5 μm (right, panels 1-4).

FIG. 6. Immunoblot analyses showing the levels of chloroplast division proteins during leaf development. PDV2 and FtsZ2-1 are detected by anti-PDV2 and anti-FtsZ2-1 antibodies, respectively. DRP5B and ARC6 levels were analyzed by anti-GFP antibodies using wild-type plants expressing GFP-DRP5B and ARC6-GFP by their respective promoters. The same amount of protein extracted from leaves corresponding to stages 1-4 (indicated in A, a sample of stage 1 including both the young emerging leaves and shoot apexes) was loaded in each lane. Three biological replicates showed the same result.

FIG. 7. Histochemical GUS staining of PDV2 promoter-GUS, FtsZ2-1 promoter-GUS and DRP5B promoter-GUS transgenic plants. 3-week-old plants or less grown on agar plates were stained. Magnified images of the centers of the rosettes are also shown (insets). ARC6 promoter-GUS transgenic plants were also prepared but we could not obtain a staining signal. Three independent transgenic lines for each promoter showed the same results. Bar=3 mm.

FIG. 8. Fluorescence microscopy showing the localization of chloroplast division proteins during leaf development. GFP-PDV1, GFP-PDV2, FtsZ-GFP, GFP-DRP5B and ARC6-GFP expressed by their respective promoters were observed in emerging (top panels) and expanding (middle panels) leaves corresponding to stages 1 and 4 in FIG. 5. GFP-PDV1, GFP-PDV2, and GFP-DRP5B were over-expressed in wild type plants and the localization was observed by fluorescence microscopy (bottom panels). In emerging (35S-DRP5B) and expanding (35S-PDV1 and 35S-PDV2) leaves corresponding to stages 1 and 4 in FIG. 5, these proteins localize at the chloroplast division site. Three independent transgenic lines for each GFP-fusion showed the same results. Bars=5 μm.

FIG. 9. Chloroplasts of the wild type and PDV1 and PDV2 over-expressers in emerging and expanding leaves corresponding to stages 1 and 4 in FIG. 5. Bar=5 μm.

FIG. 10.

(A) Chloroplasts of wild type (WT) and transgenic plants over-expressing CRF2 (35S-CRF2) in single leaf mesophyll cells. Tips of the first true leaves were cut from 3-week-old or less plants grown on agar plates. Bar=20 μm.

(B) RT-PCR analyses showing the CRF2 transcript was increased in 35S-CRF2 transgenic plants. Total RNA extracted from rosettes were used for RT-PCR. UBQ1 was used as the internal control. The number of PCR cycles was 28 for CRF2 and 26 for UBQ1. The signals were estimated by ethidium bromide staining. Two biological replicates showed the same result.

(C) immunoblot analyses comparing the levels of the chloroplast division proteins between the wild type and CRF2 over-expresser. The same amount of total protein extracted from 3-weeks-old or less russets was loaded in each lane. Rubisco large subunit stained by Ponceau S is shown as the loading control. Three biological replicates showed the same result.

FIG. 11.

(A) Effect of cytokinin treatment on the size and number of chloroplasts, Chloroplasts in single mesophyll cells of the cotyledon are shown. Wild-type seeds were germinated and grown for 10 days on agar plates with (+BA) or without (−BA) 5 μM 6-benzyladenine. Bar=20 μm.

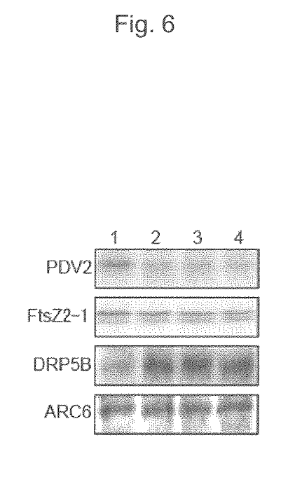

(B) immunoblot analyses comparing the levels of the chloroplast division proteins between wild type seedlings germinated on medium with (+BA) or without (−BA) 6-benzyladenine. The same amount of total protein extracted from 10-day-old seedlings was loaded in each lane. PDV2, FtsZ2-1, GFP-DRP5B, and ARC6-GFP were detected as in FIG. 6. Three biological replicates showed the same result.

FIG. 12. Number of chloroplasts per chloronema cells of the wild-type (WT) and Pp PDV2-1 over-expresser (Pp PDV2-OX) (n=30 cells). In P. patens, protonema cells are classified into chloronema and caulonema. Chloronema, which contains round chloroplasts, develops to caulonema, which contains spindle-shaped chloroplasts. An increase in number of chloroplasts was also observed in caulonemal cells of the Pp PDV2-1 over-expresser. RT-PCR analyses showing the Pp PDV2-1 transcript is increased in the transgenic line. Total RNA extracted from protonemal colonies was used for RT-PCR. Pp ACTIN3 was used as the internal control. The number of PCR cycles was 28 for Pp PDV2-1 and 28 for Pp ACTIN3. The signals were estimated by ethidium bromide staining. The same results were obtained in four independent transformants. Scale bar=10 μm.

FIG. 13. Effect of cytokinin treatment on chloroplasts and expression of chloroplast division genes. RT-PCR analyses comparing transcript levels of chloroplast division genes between cells grown on medium with (+BA) or without (−BA) 6-benzyladenine. Four day-old protonemal cells were transferred onto medium with or without 5 μM: 6-benzyladenine and grown for 4 days. Pp ACTIN3 was used as the internal control. The number of PCR cycles was 28 for each gene. The signals were estimated by ethidium bromide staining. Two biological replicates showed the same result. CN, caulonema; GS, Gametophore shoot apical cell of a bud induced by cytokinin. Bars=10 μm.

### DETAILED DESCRIPTION OF THE INVENTION

This invention will be described in more detail below.

### Transformed Plants

The transformed plants of the invention are characterized by increased number of chloroplasts, the level of which is higher than that of wild types. This character of the plants is achieved by over-expressing a foreign (or exogenous) DNA coding for proteins PDV1, PDV2 and/or CRF2 or a homologue thereof in the plants.

As used herein, the term “PDV” is an abbreviation of “Plastid DiVision protein”, and is an outer envelope-spanning protein which contains coiled-coil domains on the cytosolic side (Miyagishima et al., 2006; Glynn et al., 2008) and determines the rate of chloroplast division as a land-plant specific components of the division apparatus. Two PDV1 proteins “PDV1” and “PDV2”, which are paralogues of each other (Miyagishima et al., 2006), are known. In this invention, it has been found that the level of PDV proteins determines the rate of chloroplast division in the angiosperm Arabidopsis thaliana and the moss Physcomitrella patens. The analyses also show that the PDV level is up-regulated by the plant hormone cytokinin. Acquisition of PDV proteins by the common ancestor of land plants has linked the cell differentiation program and chloroplast division, and has enabled land plant cells to change the number of chloroplasts based on cell differentiation.

As used herein, the term “CRF2” is an abbreviation of “Cytokinin Response Factor 2”, and is a putative cytokinin responsive transcription factor which, together with other paralogous CRF proteins, mediates transcriptional responses to the plant hormone cytokinin (Rashotte et al., 2006).

As used herein, the term “over-expressing”, “over-expressed” or “over-expression” means that an expression level of the PDV1, PDV2 and/or CRF2 proteins or homologues thereof in the transformed plants of the invention is higher than that in wild types which has no foreign PDV1, PDV2 and/or CRF2 and/or homologues thereof.

As used herein, the term “homologue” means a protein from any plants other than the angiosperm Arabidopsis thaliana and the moss Physcomitrelia patens, which protein comprises an amino acid sequence homologous to that of PDV1, PDV2 or CRF2 protein and has more increased number of chloroplasts than wild type.

As used herein, the term “foreign” means that PDV1, PDV2 and/or CRF2 proteins or homologue thereof is not endogenous. In other words, the PDV1, PDV2 and/or CRF2 or homologues coding DNA is introduced exogenously into plants.

In this invention, the PDV1, PDV2 and/or CRF2 or homologues thereof may be mutated as long as the mutants have more increased number of chloroplast than wild type when they are expressed in plants.

In this invention, the plants include all land plants having chloroplasts. Examples of such land plants include, but are not limited to, mosses, ferns, gymnosperm and angiosperm (including dicotyledonous plants, monocotyledonous plants, tree plants). Specifically, examples of plants include species belonging to orders such as Jungermanniales, Marchantiales, Enbryales, Filicales, Cycadales, Ginkgoales, Taxodiales, Pdocarpales, Ephedrales, Magnoliales, Laurales, Capparales, Fabales, Poales, Uricales, Fagales, Caryophyilales, Theales, Salicales, Ericales, Rosales, Myrtales, Sapindales, Apiales, Saponales, Lamiales and Asterales, and more specifically, include species such as Arabidopsis thaliana, Brassica napus, Brassica oleracea var. italica, Raphanus sativus L., Brassica oleraceae var. botrytis, Brassica oleracea var. capitata, Brassica rapa var. glabra, Oryza sativa, Triticum aestivum, Hordeum vulgare, Zea mays, Glycine max, Lotus corniculatus var. japonicus, Solanum lycopersicum, Solanum melongena, Solanum tuberosum L., Allium fistulosum, Allium cepa, Allium sativum, Spinacia oleracea, Saccharum officinarum, Eucalyptus, Populus, Elaeis gunineensis, Wasabia japonica, Allium tuberosum, etc.

In this invention, progeny of the transformed plants is also encompassed. Progeny includes second generation, third generation, and further subsequent generations. The progeny may generally be generated by callus, or alternatively by crossing the transformed plant with wild type. The progeny of the invention is characterized by increased number of chloroplast as compared with wild type.

### PDV1 and PDV2 and Homologues Proteins and Mutant Proteins Thereof

The amino acid and nucleotide sequences of PDV1 and PDV2 of Arabidopsis thaliana or homologue proteins thereof and DNAs encoding them are available from known databases such as NCBI GenBank (USA), EMBL (Europe), etc. For example, GenBank accession numbers of PDV1 and homologues are as follows: A. thaliana PDV1 (protein: NP_{—}200140.1/GI:15238704; DNA: NM_{—}124707.3 GI:145359195; Locus: At5g53280), Vitis vinifera PDV1 (CA069353/gi:157360164), Populus triehocarpa PDV1 (ABK04742/gi:118485788), Oryza sativa PDV1 (NP_{—}001057719/gi:115468240, NP_{—}001046494/gi:115445429, and NP_{—}001042451/gi:115435386), A. thaliana PDV2 (Protein: NP_{—}028242/GI:18398011; DNA: NM_{—}127166.3/GI:42569065/Locus: At2g16070), V. vinifera PDV2 (CA047676/gi:157340871), O. sativa PDV2 (EAZ02618/gi:125557082), Physcomitrella patens PDV2 (Pp PDV2-1, XP_{—}001768164/gi:168031310; Pp PDV2-2, XP_{—}001763980/gi:168022907; Pp PDV2-3, XP_{—}001753151/gi:168000895; and Pp PDV2-4, XP_{—}001768164/gi:168053217).

PDV1 and PDV2 proteins of A. thaliana specifically comprise an amino acid sequence as shown in SEQ ID NOs: 1 and 2, respectively. The homologue proteins or mutant proteins of the PDV1 and PDV2 comprise amino acid sequences having an at least 20%, preferably at least 50%, more preferably at least 70-85%, yet more preferably at least 90-98% identity to the amino acid sequence of SEQ ID NO: 1 or 2 and having an activity of increasing the number of chloroplasts when compared with wild types.

The DNAs encoding PDV1 and PDV2 of A. thaliana specifically comprise a nucleotide sequence as shown in SEQ ID NOs: 4 and 5. The homologue proteins, or mutant proteins of the PDV1 and PDV2 comprise: (i) nucleotide sequences having an at least 20%, preferably at least 50%, more preferably at least 70-85%, yet more preferably at least 90-98% identity to the nucleotide sequence of SEQ ID NO: 4 or 5; or (ii) nucleotide sequences capable of hybridizing with a nucleotide sequence complement to the nucleotide sequence of SEQ ID NO: 4 or 5 under stringent conditions, wherein the nucleotide sequences (i) and (ii) code for proteins having an activity of increasing the number of chloroplasts when compared with wild types.

### CRF2 and Homologues Proteins and Mutant Proteins Thereof

The amino acid and nucleotide sequences of CRF2 of Arabidopsis thaliana or homologue proteins thereof and DNAs encoding them are available from known databases such as NCBI GenBank (USA), EMBL (Europe), etc. For example, GenBank accession numbers of CRF2 protein and DNA encoding DRF2 of A. thaliana are as follows: CRF2 protein: NP_{—}194106.1 and NP_{—}974599.1; DNA encoding of CRF2 NM_{—}118506.2/GI:186512869 and NM_{—}202870.1; Locus: At4g23750).

CRF2 protein of A. thaliana specifically comprises an amino acid sequence as shown in SEQ ID NO: 3. The homologue proteins or mutant proteins of the CRF2 comprise amino acid sequences having an at least 20%, preferably at least 50%, more preferably at least 70-85%, yet more preferably at least 90-98% identity to the amino acid sequence of SEQ ID NO: 3 and having an activity of increasing the number of chloroplasts when compared with wild types.

The DNA encoding CRF2 of A. thaliana specifically comprises a nucleotide sequence as shown in SEQ ID NO: 6. The homologue proteins, or mutant proteins of the CRF2 comprise: (i) nucleotide sequences having an at least 20%, preferably at least 50%, more preferably at least 70-85%, yet more preferably at least 90-98% identity to the nucleotide sequence of SEQ ID NO: 6; or (ii) nucleotide sequences capable of hybridizing with a nucleotide sequence complement to the nucleotide sequence of SEQ ID NO: 6 under stringent conditions, wherein the nucleotide sequences (i) and (ii) code for proteins having an activity of increasing the number of chloroplasts when compared with wild types.

The aforementioned term “mutant” comprises one or more, preferably one or several, deletions, substitutions or additions in the amino acid or nucleotide sequences of PDV1, PDV2 and CRF2 or homologues thereof. The term “several” as used herein refers to an integer between 2 and 20, inclusive. The mutant may include either naturally occurring mutants or artificial mutants.

Where the mutant is a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gln, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

Where the mutant is a nucleic acid, the DNA encoding a mutant protein of PDV1, PDV2 or CRF2 or homologue thereof may comprise a nucleotide sequence capable of hybridizing to a complement sequence of the nucleotide sequence encoding PDV1, PDV2 or CRF2 or homologue thereof as defined above, under stringent conditions. As used herein, the stringent conditions include low, medium or high stringent conditions. An example of the stringent conditions includes hybridization at approximately 42-55° C. in approximately 2-6×SSC, followed by wash at approximately 50-65° C. in approximately 0.1-1×SSC containing approximately 0.1-0.2% SDS, where 1×SSC is a solution containing 0.15 M NaCl and 0.015 M Na citrate, pH 7.0. Wash can be performed once or more. In general, stringent conditions may be set at a temperature approximately 5° C. lower than a melting temperature (Tm) of a specific nucleotide sequence at defined ionic strength and pH.

Said mutants can be prepared using known techniques such as site-directed mutagenesis and PCR.

Also, DNAs encoding PDV1, PDV2 and CRF2 or homologues thereof may be prepared and amplified from genomic or cDNA libraries derived from organs or tissues of a plant, by using known cloning and PCR techniques. Organs include, but are not limited to, roots, stems, leaves, petals, seeds, etc., and tissues include, but are not limited to, epidermis, phloem, parenchyma, xylem, vascular bundle, palisade tissues, spongy tissues, etc.

These techniques are described in Sambrook et al., Molecular Cloning A Laboratory Manual, 1989, Cold Spring Harbor Laboratory Press, Ausubel et al., Current Protocols in Molecular Biology, 1994, joint Wiley & Sons, etc.

### Transformation

The transformed plants of the invention can be prepared by transforming the cells of plants with a vector comprising a DNA encoding PDV1, PDV2 and/or CRF2 protein or homologue thereof, which DNA is as defined above.

The transformed plant can be produced by a method comprising the following steps of:
(1) introducing a vector comprising the DNA as defined above into cells of a plant to obtain transformed cells;
(2) selecting a transformed cell, which over-expresses the DNA, from the transformed cells of step (1); and
(3) generating the transformed plant from the transformed cell of step (2).

For transformation of, basically the same or similar methods can be used. For example, transformation can be performed by methods of using viral vectors (e.g., binary bector-Agrobacterium system), particle gun, electroporation, floral dip (Clough and Bent, Plant J. 16: 735-743 (1998)), leaf disc, and the like.

In general, vectors usable for transformation of plants are binary vectors. The binary vector comprises two approximately 25-hp border sequences, i.e. right border (RB) and left border (LB) derived from Agrobacterium T-DNA. A foreign DNA can be inserted between the two border sequences, and a promoter is linked to the 5′-end of the foreign DNA.

Examples of the promoter include, but are not limited to, cauliflower mosaic virus (CaMV) 35S promoter (Jefferson, R. A. et al.: The EMBO 6:3901-3907 (1987)]), noparin synthase gene promoter (Christensen, A. H. et al.: Plant Mol. Biol. 18:675-689 (1992)), ubiquitin corn promoter, octopin synthase gene promoter, rice actin promoter, and the like. Other promoters include rd29Agene promoter, rd29B gene promoter, rd17 gene promoter, rd22 gene promoter, cor6.6 gene promoter, cor15a gene promoter, erd1 gene promoter, kin1 gene promoter, etc (JP-2008-505603A).

In the vectors, a terminator may be linked to the 3′-end of the foreign DNA. Examples of the terminator include, but are not limited to, noparin synthase gene terminator, cauliflower mosaic virus derived terminator, and the like.

The vectors may contain a selectable marker or reporter gene necessary for screening transformed cells of interest. Examples of the selectable marker include, but are not limited to, drug resistant genes such as kanamycin resistant gene (NPTII), hygromycin resistant gene (htp), biarafos resistant gene, and the like. Examples of the reporter gene include, but are not limited to, GFP (green fluorescence protein) gene, Gus (β-glucuronidase) gene

Examples of binary vectors include, but are not limited to, pBI plasmids such as pBI101, pBI101.2, pBI101.3, pBI121, pBI221, pBE2113Not, pBI2113Not, pBI2113, pGA482, pGAH, pBIG, etc., and other plasmids such as pLGV23Neo, pNCAT, pMON200, pH35GS containing GATEWAY (Kubo et al., 2005. Genes & Dev. 19: 1855-1860), etc.

When the binary vector-Agrobacterium system is used, the method comprises: providing cells, calli, or tissues from a plant or alga; and infecting them with Agrobacterium containing binary vector, thereby introducing the above-defined DNA into the cells of a plant.

Normally used as Agrobacterium are Agrobacterium tumefaciens strains, such as C58, LBA4404, EHA101, EHA105, C58C1RifR, etc.

Media for transformation are MS medium, B5 medium, DKN medium, Linsmaier & Skoog medium, etc. In general, to these basal media may be added 1-5% succaride such as maltose, sucrose, glucose, or sorbitol, and a solidification agent consisting of 0.2-1% polysuccaride such as agar or gellan gum. Media may further contain auxins or cytokinins, such as casamino acid, abscisic acid, kinetin, 2,4-D, or indole acetic acid; antibiotics such as kanamycin, hygromycin, or carbenicillin; acetosyringone; or mixtures thereof. Acetosylingone, which is a phenolic compound, can be used effectively for transformation of monocotyledonous plants. Preferred pH of the medium is pH 5-6.

In transformation with the binary vector-Agrobacterium system, Agrobacterium is cultured at approximately 25° C. for about 4 days in the dark, and then plant callus or tissue (e.g., leaf piece, root, stem piece, or growing point) is dipped in the culture medium of Agrobacterium for several minutes, and after removal of water, the callus or tissue is co-cultured with Agrobacterium on a solid medium. The transformed callus or tissue can be selected for selectable marker (e.g., by culturing them in a medium containing antibiotic) or reporter (e.g., by detecting a fluorescence). The callus can redifferentiate into seedlings on a redifferentiation medium. The tissue may be transformed directly, or alternatively protoplasts may be prepared from the tissue, followed by induction of calli, which are subsequently redifferentiated into seedlings. After the roots are developed, the seedlings are transferred to soil for reproduction of plant. From the reproduced plant, seeds are collected in order to obtain transformed plants (or transgenic plants). Further, progeny can be obtained from the transformed plants through cultivation. The progeny with increased the number of chloroplast per cell, also falls within the scope of the invention.

Host for use in transformation are as described above, and thus they include all land plants having chloroplasts.

### EXAMPLES

### Experimental Procedures

The Examples of this application were performed by using the following materials in accordance with the following methods.

### (Plant Materials and Growth Conditions)

A. thaliana (Col-0) was used as wild type. A. thaliana seeds were surface-sterilized, sown on Murashige and Skoog (MS) plates, and stratified at LIT for 48 h in the dark before germination. Plants were grown in controlled-environment chambers with 16 h of light (100 μmol/m²s) and 8 h of dark at 20″C. For cytokinin treatment, sterilized seeds were sown on MS plates supplemented with 5 μM 6-benzyladenine (BA) and grown for 10 days alter germination. The pdv1 and pdv2 mutants used in this study are pdv1-1 (has a premature stop codon close to the start codon) and pdV2-1 (T-DNA is inserted into the first intron), respectively (Miyagishima et al., 2006).

Physcomitrella patens (Hedew.) Bruch & Schimp subsp patens Tan was grown in controlled-environment chambers with 16 h of light (100 μmol/m²s) and 8 h of dark at 20° C. on the minimal medium (BCD medium) supplemented with 0.5% glucose, 1 mM CaCl₂ and 5 diammonium (+)-tartrate agar plate as described (Nishiyama et al., 2000). For cytokinin treatment, protonema grown on BCD medium were transferred and then grown on BCD medium containing 5 μM. BA for 4 days.

(Analyses of A. thaliana Full-Length cDNA Over-Expressing Gene Hunting System (FOX) Lines)

Seeds of 10,000 or less independent FOX lines (Ichikawa et al., 2006) (Col-0 background) were separated to 200 pools, in which 50 or less independent lines were mixed in each pool. One hundred seeds from each pool were germinated and grown for 3 weeks on MS plates. Tips from expanding leaves were put on a glass slide without fixation, covered with a cover slip, and were observed with Nomarski optics. The inserted cDNA of the lines, which contain larger number chloroplasts than wild type, were amplified by T-DNA-specific primers FOX.F (SEQ ID NO:7) and FOX.R (SEQ ID NO:8) and sequenced by primer FOX-seq (SEQ ID NO:9).

(Constructing Over-Expression and Fusion Constructs and Generating Transgenic A. thaliana)

For over-expression of PDV1, PDV2, and CRF2, fragments containing respective open reading frames (orf) franked by 0.1 or less kb 5′ upstream sequences were amplified by primers: PDV1-ox.F (SEQ ID NO:10) and PDV1-ox.R (SEQ ID NO:11) for PDV1, PDV2-ox.F (SEQ ID NO:12) and PDV2-ox.R (SEQ ID NO:13) for PDV2, and, CRF2-ox.F (SEQ NO:14) and CRF2-ox.R (SEQ ID NO:15) for CRF2 (see, Table 1). The amplified PDV2 and CRF2 fragments were digested with XbaI and SmaI (the recognition sequences of these enzymes are underlined in the sequences of respective primers), and the PDV1 fragment was digested with NheI and SmaI. The digested products were inserted between XbaI and blunting SacI sites (downstream of cauliflower mosaic virus 35S promoter) of pBI121 vector (conferring resistance to kanamycin; Clontech).

For over-expression of GFP-DRP5B, DRP5B promoter of DRP5B promoter-GFP-DRP5B construct (Miyagishima et al., 2006) was replaced by 355 promoter amplified from pBI121 vector. The generated constructs were transformed into wild type.

For expression of GFP-PDV2 by the PDV2 promoter, two unique restriction sites were added between the PDV2 promoter and the start codon by overlap-extension PCR. A 1.2-kbp 5′ upstream sequence of PDV2, including the start codon, was amplified by primers GFP-PDV2.F1 (SEQ ID NO:16) and GFP-PDV2.R1 (SEQ ID NO:17). A PDV2 on flanked by a 60 bp 3′ downstream sequence was amplified by primers GFP-PDV2.F2 (SEQ ID NO:18) and GFP-PDV2.R2 (SEQ ID NO:18). These two amplified fragments were mixed and fused by PCR using primers GFP-PDV2.F1 (SEQ NO:16) and GFP-PDV2.R2 (SEQ ID NO:18). The fused fragment was cloned into pGEM-T Easy (Promega). An orf of GFP was amplified by primers 5′-GFP-BamHI.F (SEQ ID NO:20) and GFP-KpnI.R (SEQ ID NO:21), digested with BamHI and KpnI, and cloned between a 5′ flanking region and an orf of PDV2. The resulting PDV2 promoter-GFP-PDV2 fusion was excised with NotI and then transferred into pMLBART (conferring resistance to glufosinate ammonium; Vitha et al., 2001). The construct was transformed into the wild type.

For expression of GFP-PDV1 by the 35S promoter, GFP-PDV1 fusion (Miyagishima et al., 2006) was cut out with BamHI and EcoRI and inserted into BamHI and EcoRI sites of pBI121. For expression of GFP-PDV2 by the 35S promoter, GFP-PDV2 was amplified by using the PDV2 promoter-GFP-PDV2 fusion described above as template by primers PDV2-ox.F (SEQ ID NO:12) and PDV2-ox.R (SEQ ID NO:13). The amplified fragment was digested with XbaI and SmaI, and inserted into XbaI and blunted SacI sites of pBI121. The generated construct was transformed into the wild type.

For expression of ARC6-GFP by the ARC6 promoter, ARC6 orf franked by 1.0 or less kb 5′ upstream sequence was amplified by primers ARC6-GFP.F (SEQ ID NO:22) and ARC6-GFP.R (SEQ ID NO:23) and was cloned into pGEM-T Easy. An orf of GFP(S65T) was amplified by primers GFP-KpnI.F (SEQ ID NO:24) and GFP-SacII.R (SEQ ID NO:25), digested with KpnI and SacII, and cloned downstream of the ARC6 orf. The resulting ARC6 promoter-ARC6-GFP fusion was excised with NotI and then transferred into pMLBART. The generated construct was transformed into the wild type.

FtsZ promoter-GFP-FtsZ (Nakanishi et al., 2009) and DRP5B promoter-GFP-DRP5B (Miyagishima et al., 2006) transformants were previously generated as described.

To create promoter-GUS (beta-glucuronidase) fusion, 0.6-1.5 kb 5′ regions of PDV2, FtsZ2-1, and DRP5B, respectively, were amplified by primers: PDV2-GUS.F (SEQ ID NO:26) and PDV2-GUS.R (SEQ ID NO:27) for PDV2, FtsZ2-1-GUS.F (SEQ ID NO:28) and FtsZ2-1-GUS.R (SEQ ID NO:29) for FtsZ2-1, and DRP5B-GUS.F (SEQ ID NO:30) and DRP5B-GUS.R (SEQ ID NO:31) for DRP5B. PDV2 fragment was digested with BamHI and FtsZ2-1 and DRP5B fragments were digested with XbaI (the recognition sequences of these enzymes are underlined in the sequences of respective primers). These fragments were cloned into BamHI site or XbaI site of pBI101 (conferring resistance to kanamycin; Clontech). The constructs were transformed into the wild type.

All constructs were transferred to Agrobacterium tumefaciens GV3101 and introduced into A. thaliana as described (Clough and Bent, 1998). T1 plants were selected by resistance to glufosinate or kanamycin as described (Miyagishima et al., 2006). In order to over-express both PDV1 and PDV2, a PDV1 over-expresser line was crossed with a PDV2 over-expresser line and the next generation was examined. In order to express GFP-DRP5B and ARC6-GFP in CRF2 over-expresser, GFP-DRP5B and ARC6-GFP expressing wild type, respectively, were crossed with 35S-CRF2 plants and the next generation was used for further analyses. Before using each transgenic plant for further analyses, the existence of respective transgene(s) was confirmed by PCR analyses.

(Construction and Generating Transgenic P. patens)

For over-expression of P. patens PDV2-1 (GI 168031309), a fragment containing the orf was amplified by primers Pp_PDV2-1-ox.F (SEQ ID NO:34) and Pp_PDV2-1-ox.R (SEQ. ID NO:35) from genomic DNA. (the Sinai sites are underlined). The amplified product was digested with SmaI and was inserted into SmaI site of the expression vector pPpMADS2-7113 with E7113 promoter (Mitsuhara et. al., 1996). The construct was cut out by NotI and was introduced into the protoplasts of P. patens by polyethylene glycol-mediated protocol (Nishiyama et al., 2000). The protoplasts were grown in the regeneration medium for 3 days and then transferred onto BCDAT medium (Nishiyama et al., 2000) containing 50 μg ml⁻¹ G418 to select transformants. The selected plants were transferred onto a medium without G418 and allowed to grow for 1 week. Then, they were transferred again onto the selection medium.

### (Microscopy)

For observation of chloroplasts in A. thaliana leaf cells, tips from expanding leaves from 3-week-old or less plants (or other stages where indicated) grown on MS plates were cut and fixed with 3.5% glutaraldehyde and then incubated in 0.1 M Na₂-EDTA, pH 9.0, for 15 min at 50° C. Chloroplasts of P. patens in protonemal cells were observed without fixation. Samples were observed with Nomarski optics. GFP fluorescence was visualized without fixation in young and expanding leaves from 3-week-old or less plants.

### (Analyses of Gene Expression by RT-PCR and GUS Staining)

Total RNA of A. thaliana was extracted from 3 or less weeks-old plants using an RNeasy Mini Kit (Qiagen). Total RNA of P. patens was extracted from 8 days-old colonies using RNeasy Mini Kit. DNase-treated. RNA (1 μg) was reverse-transcribed with oligo dT (15 nucleotides) primer, and resulting cDNA was used as template for PCR. Before comparison of expression levels, the linear range of the amplification was confirmed by comparing different cycles of amplification. PCR was performed using primer sets as follows: PDV1-rt.F (SEQ ID NO:36) and PDV1-rt.R (SEQ ID NO:37) for A. thaliana PDV1, CRF2-rt.F (SEQ ID NO:38) and CRF2-rt.R (SEQ ID NO:39) for A. thaliana CRF2, Pp_PDV2-1-rt.F (SEQ ID NO:42) and Pp_PDV2-1-rt.R (SEQ ID NO:43) for P. patens PDV2-1, Pp_PDV2-2-rt.F (SEQ ID NO:44) and Pp_{—} PDV2-2-rt.R (SEQ ID NO:45) for P. patens PDV2-2, Pp_PDV2-3-rt.F (SEQ ID NO:46) and Pp_PDV2-3-rt.R (SEQ ID NO:47) for P. patens PDV2-3, Pp_PDV2-4-rt.F (SEQ ID NO:48) and Pp_PDV2-4-rt.R (SEQ ID NO:49) for P. patens PDV2-4, Pp_FtsZ1-1-rt.F (SEQ ID NO:50) and Pp_FtsZ1-1-rt.R (SEQ ID NO:51) for P. patens FtsZ1-1 (GI 168056460), Pp_FtsZ1-2-rt.F (SEQ ID NO:52) and Pp_FtsZ1-2-rt.R (SEQ ID NO:53) for P. patens FtsZ1-2 (GI 168033106), Pp_FtsZ2-1-rt.F (SEQ ID NO:54) and Pp_FtsZ2-1-rt.R (SEQ ID NO:55) for P. patens FtsZ2-1 (GI 168026867), Pp_FtsZ2-2-rt.F (SEQ ID NO:56) and Pp_FtsZ2-2-rt.R (SEQ ID NO:57) for P. patens FtsZ2-2 (GI 168028518), Pp_FtsZ3-rt.F (SEQ ID NO:58) and Pp_FtsZ3-rt.R (SEQ NO:59) for P. patens FtsZ3 (GI 168025379), Pp_DRP5B-rt.F (SEQ ID NO:60) and Pp_DRP5B-rt.R (SEQ ID NO:61) for P. patens DRP5B (GI 76880153), and Pp_ARC6-rt.F (SEQ NO:62) and Pp_ARC6-rt.R (SEQ ID NO:63) for P. patens ARC6 (GI 168052683). As a control for A. thaliana, an UBQ1cDNA was amplified by primers UBQ1-rt.F (SEQ ID NO:40) and UBQ1-rt.R (SEQ ID NO:41). As a control for P. patens, ACTIN3 (=DNA was amplified by primers Pp_ACTIN3-rt.F and Pp_ACTIN3-rt.R.

GUS expression analyses were performed as described (Jefferson et al., 1987) with some modifications. A thaliana grown on MS plates were soaked in the GUS assay solution (0.5 mg/ml 5-bromo-4-chloro-3-indolylglucronide, 0.5 mM K₃Fe(CN)₆, 0.5 mM K₄Fe(CN)₆, 0.1% (v/v) Triton X-100, and 100 mM Pi-buffered saline) and incubated at 37° C., overnight for PDV2-GUS and for 3 h for the others. Then the samples were washed by 70% ethanol and soaked in ethanol and acetic acid mixture (6:1, by volumes) to stop reaction and remove chlorophylls,

### (Antibodies and Immunoblotting)

The polyclonal rabbit antibodies against A. thaliana PDV2 were raised against recombinant PDV2. The full length PDV2 coding region was amplified from A. thaliana cDNA by primers His-PDV2.E (SEQ ID NO:32) and His-PDV2.R (SEQ ID NO:33) and was cloned into pET100 expression vector (Invitrogen). Six-His fusion proteins were expressed in Escherichia coli (Rosetta 2 DI 3; Novagen), purified and injected to rabbits to stimulate antibody production. The antisera were purified by N-hydroxysuccinimide-activated column (HiTrap NHS-activated HP; GE Healthcare) conjugated with the recombinant PDV2 according to the manufacturer's protocol.

Three-weeks-old or less plants grown on MS plates were frozen in liquid nitrogen, ground with pestles, and homogenized in extraction buffer (50 mM Tris, pH 7,5, 2 mM MgCl₂, 5 mM EDTA, and a protease inhibitor mixture [P2714; Sigma]). The homogenate was filtered through Miracloth (Calbiochem). Protein concentration of the homogenate was determined and then subjected to immunoblotting. To detect proteins during leaf development, leaves were separately collected in order of size, and extracted. Samples containing 50 μg of proteins were subjected to immunoblot analyses.

Immunoblotting assays were performed as previously described (Nakanishi et al., 2009). Anti-PDV2 antibodies, anti-FtsZ2-1 antibodies (Nakanishi et al., 2009), and anti-GFP mouse monoclonal antibody (JL-8; Invitrogen) were diluted 1:20,000, 1:10,000 and 1:1,000, respectively. The primary antibody was detected by horseradish peroxidase-conjugated goat anti-mouse or anti-rabbit antibody diluted at 1:20,000. The signal was detected by SuperSignal West Pico Chemiluminescent Substrate (Thermo Scientific) and VersaDoc 5000 imaging system (Bio-Rad). We confirmed that the signal was not saturated by comparison of the images by longer and shorter exposure time.

### (Phylogenetic Analyses)

Deduced amino acid sequences of PDV1 and PDV2 homologs encoded by the 13 genes (GI numbers are indicated above) were collected by BLAST searches. The sequences were aligned by Clustal X 2.0 (Larkin et al., 2007), manually refined and 144 amino acid residues were used for the phylogenetic analyses. Maximum likelihood trees were constructed using RaxML 7.0.4 (Stamatakis, 2006) with 100 replicates using the WAG matrix of amino acid replacements assuming a proportion of invariant positions and four gamma-distributed rates (WAG+I+gamma model). Bayesian inference was performed with the program MrBayes version 3.1.2 (Ronquist and Huelsenbeck, 2003) using the WAG+1+gamma model. For the MrBayes consensus trees, 1,000,000 generations were completed with trees collected every 100 generations.

### Example 1

In order to identify the factors that modulate the rate of chloroplast division, genes that accelerate chloroplast division when the genes are over-expressed were searched.

Seeds of about 10,000 independent FOX lines (Full-length cDNA Over-expressing gene hunting system lines; Ichikawa et al., 2006) (Arabidopsis thaliana Columbia-0 background) were screened. In each FOX line, a full length cDNA is expressed under the cauliflower mosaic virus 35S promoter (Ichikawa et al., 2006). Said about 10,000 independent FOX lines were separated to 200 pools, in which 50 or less independent lines were mixed in each pool. Bach seed was surface-sterilized, shown on Murashige and Skoog (MS) plates, and stratified at 4° C. for 48 h in the dark before germination. One hundred seeds from each pool were germinated and grown for 3 weeks on MS plates. Plants were grown in controlled-environment chambers with 16 h of light (100 μmol/m²s) and S h of dark at 20° C. Tips from expanding leaves were put on a glass slide without fixation, covered with a cover slip, and were observed with Nomarski optics. As a result, six independent lines which grow normally and contain a larger number of chloroplasts in the expanded leaves than the wild type were isolated. Of these, 5 lines contained a 35S promoter-PDV2 transgene and the immunoblot analysis using anti-PDV2 antibodies showed that PDV2 protein was over-expressed in the line.

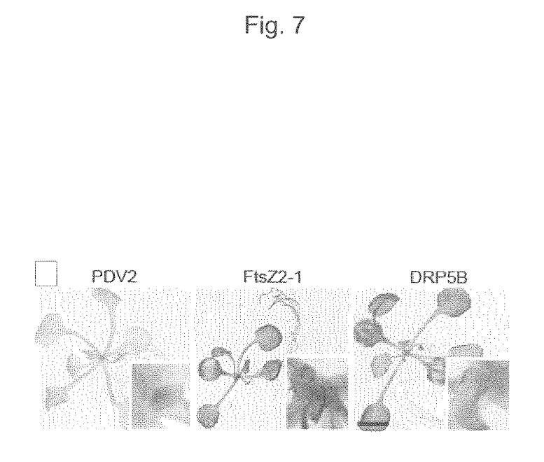

To further confirm whether the phenotype is linked to the over-expression of PDV2, we prepared a PDV2 over-expresser by a newly-constructed 35S-PDV2 transgene. For over-expression of PDV2, fragments containing respective open reading frames (orf) franked by 0.1 or less kbp 5′ upstream sequences were amplified by primers: PDV2-ox.F (SEQ NO:12) and PDV2-ox.R (SEQ ID NO:13) for PDV2 in Table 1. The amplified PDV2 fragment was digested with XbaI and SmaI (the recognition sequences of these enzymes are underlined in the sequences of respective primers in Table 1). The digested products were inserted between XbaI and blunting SacI sites (downstream of cauliflower mosaic virus 35S promoter) of pBI121 vector (conferring resistance to kanamycin; Clontech). The results are shown in FIG. 7. The phenotype of the transgenic plant displayed the same phenotypes as the isolated FOX lines.

TABLE 1

| | | |
|---|---|---|
| Oligonucleotide Name | Sequence (5′-3′) | SEQ ID NO. |

For identification of overexpressing cDNA in FOX mutant

| | | |
|---|---|---|
| FOX.F | GTACGTATTTTTACAACAATTACCAACAAC | 7 |
| FOX.R | GGATTCAATCTTAAGAAACTTTATTGCCAA | 8 |
| FOX-seq | CCCCCCCCCCCCD | 9 |

For construction of overexpresser

| | | |
|---|---|---|
| PDV1-ox.F | AGTGCTAGCACAATTAAAGTGAGAATTTTC | 10 |
| PDV1-ox.R | AGTCCCGGGCTGGGGTGCGAATGGGCTGAT | 11 |

For construction of PDV2 overexpresser

| | | |
|---|---|---|
| PDV2-ox.F | ATGTCTAGAAAACACTCCAAATTACTTGTC | 12 |
| PDV2-ox.R | CATCCCGGGATGTCAGGCAATGAACATTAC | 13 |

For construction of CRF2 overexpresser

| | | |
|---|---|---|
| CRF2-ox.F | GCTTCTAGATCCTCTTCTTATCTCTCTCTC | 14 |
| CRF2-ox.R | GTACCCGGGTTAAACAGCTAAAAGAGGATC | 15 |

For construction of GFP-PDV2 fusion

| | | |
|---|---|---|
| GFP-PDV2.F1 | GCTTAGAAACTCCGACAAGAA | 16 |
| GFP-PDV2.R1 | GGTACCCCGGGATCCAATTAACCCGATGCC | 17 |
| GFP-PDV2. F2 | GGATCCCGGGGTACCATGGAAGACGAAGAA | 18 |
| GFP-PDV2.R2 | ACCAAGTGGTATAAGTTGGGA | 19 |
| GFP-BamHI.F | GGATCCATGGTGAGCAAGGGCGAGGAG | 20 |
| GFP-KpnI.R | TTTGGTACCTCCTCCTCCCTTGTA | 21 |

For construction of ARC6-G FP fusion

| | | |
|---|---|---|
| ARC6-GFP.F | TTCATGGTAATTATTCTATCACTATTCAAG | 22 |
| ARC6-GFR.R | GGTACCTGATGCAAGAACAGAGCCTTCAGT | 23 |
| GFP-KpnI.F | GGGGTACCGGAGGAGGAGGAATGGTGAGCAAGGGCGAGG | 24 |
| GFP-SaclI.R | TCCCCGCGGTTACTTGTACAGCTCGTCCATGC | 25 |

For construction of GUS fusions

| | | |
|---|---|---|
| PDV2-GUS.F | AAAGGATCCGCTTAGAAACTCCGACAAGAA | 26 |
| PDV2-GUS.R | TTTGGATCCAATTAACCCGATGCCTTCTTC | 27 |
| FtsZ2-1-GUS.F | ATGTCTAGAGACGCACTGTCACTGCTACGG | 28 |
| FtsZ2-1-GUS.R | ATGTCTAGAAGTAAAACACGGTGAAACGTA | 29 |
| DRP5B-GUS.F | ATCTCTAGATTCATGTTTTTTCCCCTGGTG | 30 |
| DRP5B-GUS.R | TACTCTAGACGTCACCGATTTTGCTGATAC | 31 |

For construction of His-tagged PDV2

| | | |
|---|---|---|
| His-PDV2.F | CACCATGGAAGACGAAGAAGGCATC | 32 |
| His-PDV2.R | TCAACCGTATCCGTAAGTTAC | 33 |

For construction of Pp_PDV2-1 overexpresser

| | | |
|---|---|---|
| Pp_PDV2-1-ox.F | ATGCCCGGGATTAGGTGTGGGGGTAGGATC | 34 |
| Pp_PDV2-1-ox.R | ATGCCCGGGTCAGCCGCGGCCATGCAGGAC | 35 |

For RT-PCR

| | | |
|---|---|---|
| PDV1-rt.F | ACCGCTCTTGAGAACCTTGA | 36 |
| PDV1-rt.R | GAGGCAAAGGCGTTGATTAC | 37 |
| CRF2-rt.F | GTAGTCAAGGAAGAGCCATCCATGAC | 38 |
| CRF2-rt.R | CATGTGCCAGCTGGATAATCCGGATC | 39 |
| UBQ1-rt.F | GGCCAAGATCCAAGACAAAG | 40 |
| UBQ1-rt.R | GTTGACAGCTCTTGGGTGAA | 41 |
| Pp_PDV2-1-rt.F | CAAGTGGTGGAGGAAGCGATGACATTC | 42 |
| Pp_PDV2-1-rt.R | GTGCGTCGTTGTGCTCATATCAGCA | 43 |
| Pp_PDV2-2-rt.F | ATCGAGCTGACAAAATGCTAAC | 44 |
| Pp_PDV2-2-rt.R | GTAATAAGACAGGCTTTACTGTTG | 45 |
| Pp_PDV2-3-rt.F | CAGTGAACACACAACCATCTA | 46 |
| Pp_PDV2-3-rt.R | TTGTGTTCAGTGAATGCTAGG | 47 |
| Pp_PDV2-4-rt.F | GAAAGCAGAAGAGTTCTGTTG | 48 |
| Pp_PDV2-4-rt.R | CACTAACAATACTTTTCCTGTG | 49 |
| Pp_FtsZ1-1-rt.F | AGATGTTAGAGCTGTAATGAG | 50 |
| Pp_FtsZ1-1-rt.R | TGGCAATAATCGTTACATGGAT | 51 |
| Pp_FtsZ1-2-rt.F | TGCTGAAGAAGCTGCAATTCA | 52 |
| Pp_FtsZ1-2-rt.R | CAAGTTGAGAGTTGATTGCTTC | 53 |
| Pp_FtsZ2-1-rt.F | GAGGTAAATGCTGCAGCAGAGGTGA | 54 |
| Pp_FtsZ2-1-rt.R | TGGTATATTGATCGCAGAGCCATTG | 55 |
| Pp_FtsZ2-2-ft.F | CAGCATTGAGTGCCATTCAGTC | 56 |
| Pp_FtsZ2-2-ft R | GCTTAATAATGATGAGCAATCC | 57 |
| Pp_FtsZ3-rt.F | GTGTTCGAACTGTAGACGATG | 58 |
| Pp_FtsZ3-rt.R | GCTTTCAGAGTATCGATTGAG | 59 |
| Pp_DRP5B-rt.F | ATGTTGCTCAGGTTCGTAGAGCATTG | 60 |
| Pp_DRP5B-rt.R | TAGAGGGAACATCATGGTGTTCCAGG | 61 |
| Pp_ARC6-rt.F | CAGCCATATCATGGGTAGGACCAAGT | 62 |
| Pp_ARC6-rt.R | CAAGAATTCAGTCGATGACGGCTCTG | 63 |
| Pp_ACTIN3-rt.F | CGGAGAGGAAGTACAGTGTGTGGA | 64 |
| Pp_ACTIN3-rt.R | ACCAGCCGTTAGAATTGAGCCCAG | 65 |

Underlines indicate restriction sites.

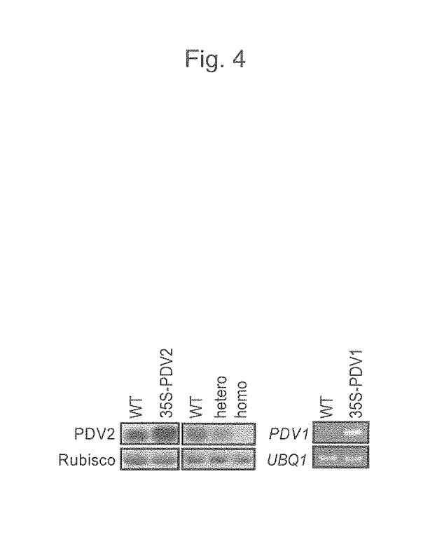

An immunoblot analysis showed that PDV2 protein is over-expressed in the line compared to the wild type (FIG. 4) and These results indicate that PDV2 over-expression accelerates chloroplast division.

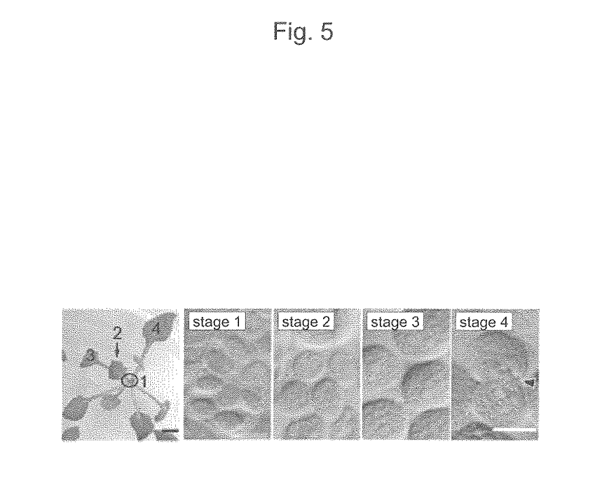

Because previous studies showed that PDV2 mediates the recruitment of DRP5B to the chloroplast division site together with PDV1, a protein paralogous to PDV2 (Miyagishima et al., 2006), the effect of PDV1 over-expression by the 35S promoter on chloroplast division was also examined. Leaf cells of the PDV1 over-expresser contained a larger number of smaller chloroplasts than the wild type (FIG. 5). When both PDV1 and PDV2 were simultaneously over-expressed, the number of chloroplasts further increased compared to the single gene over-expressers (FIG. 6). These results indicate that PDV1 and PDV2 independently accelerate chloroplast division when the genes are over-expressed.

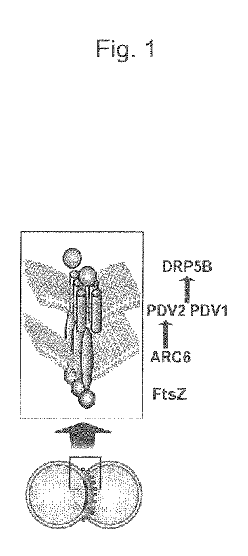

It was previously reported that the pdv2/PDV2 heterozygous T-DNA insertional mutant contains a smaller number of chloroplasts than the wild type (Miyagishima et al., 2006). The phenotype of pdv2/PDV2 is intermediate between those of the wild-type and pdv2/pdv2 plants (Miyagishima et al., 2006) (FIGS. 1B to 1D). By immunoblot analysis, we confirmed that PDV2 is absent in pdv2/pdv2, and found that the PDV2 level is reduced in pdv2/PDV2 compared to the wild type (FIG. 1D). Taken together, the above results indicate the levels of PDV1 and PDV2 positively correlate with the rate of chloroplast division. In contrast, over-expression of DRP5B had no effect on chloroplast division. Previous studies showed that over-expression of FtsZ1, FtsZ2 (Stokes et al., 2000) or ARC6 (Vitha et al., 2003) in addition to some other proteins related to the division machinery impairs chloroplast division (Colletti et al., 2000; Itoh et al., 2001; Raynaud et al., 2004), resulting in giant chloroplasts. The acceleration of chloroplast division has not been observed in the over-expression or disruption of genes that are reported to be involved in the division process. Taken together, it is suggested that levels of PDV1 and PDV2 determine the rate of chloroplast division.

### Example 2

Since these changes in the PDV levels were caused by the expression of transgenes or by mutations, it was examined whether the levels of PDV1 and PDV2 translated from endogenous genes actually change so as to modulate the chloroplast division rate in the wild-type plant. The levels of PDV and other chloroplast division proteins during leaf development were examined.

The results are shown in FIG. 5. During leaf development cells elongate and chloroplasts divided without cell division while majority of leaf cells are formed by cell division in young emerged leaves. During leaf development, frequency of chloroplasts with division site constriction decreases while the size of chloroplasts increases as the leaf gets older (Possingham and Lawrence, 1983; Boffey and Lloyd, 1988; Pyke, 1999) (FIG. 5).

Immunoblot analyses showed that the PDV2 level is highest in the apical meristem and young emerging leaves, and decreases during leaf development (FIG. 6). In contrast, the DRP5B level increased, but the FtsZ2-1 (the antibodies are specific to FtsZ2-1 of three FtsZ proteins of A. thaliana; Suzuki et al., 2009) and ARC6 levels remained constant during leaf development (FIG. 6).

Promoter-GUS fusion assays also showed that the activity of the PDV2 promoter is highest around the shoot apical meristem, in contrast to the FtsZ and DRP5B promoters (FIG. 7). These results indicate that the level of PDV2, but not that of FtsZ, ARC6, and DRP5B, decreases during leaf development.

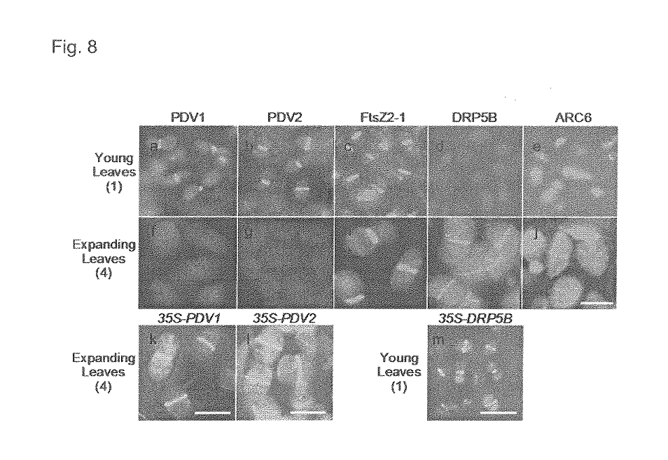

To compare the levels of the chloroplast division proteins at the chloroplast division site, GFP-fusion proteins were expressed by their respective promoters and observed by fluorescence microscopy. Consistent with the results of the immunoblot and GUS analyses, strong fluorescence signals of GFP-PDV1 and GFP-PDV2 at the chloroplast division site was clearly observed in young, emerging leaves (FIGS. 8 a and b), but the signals were hardly detected in older, expanding leaves (FIGS. 8 f and g). In contrast, the FtsZ-GFP and ARC6-GFP signals at the division site were observed in both young and older leaves (FIGS. 8 c, e, h and J). Strong GFP-DRP5B signals at the division site were observed in older leaves (FIG. 8 i), but hardly at all in young emerging leaves (FIG. 8 d). Despite the absence of signals strong enough to allow the observation of GFP-PDV1 and GFP-PDV2 in expanding leaves (FIGS. 8 k and l), or GFP-DRP5B in young emerging leaves (FIG. 8 m), the signals of these GFP fusion proteins at the chloroplast division site were detected throughout leaf development when the proteins were over-expressed. Therefore, the barely detectable GFP signals by fluorescence microscopy (FIG. 8) and faint but still detectable signals by immunoblot analyses (FIG. 6) are probably indicative of low levels of the proteins in the division apparatus, instead of the absence of these proteins.

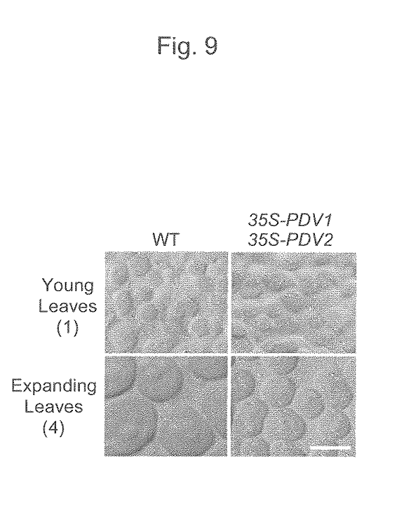

The above results indicate that the PDV levels in the chloroplast division apparatus, but not FtsZ, ARC6, or DRP5B, decrease during leaf development, suggesting that the stoichiometric relationship between the components of the division apparatus changes during chloroplast development. Given the results of over-expression and the reduction of PDV levels (FIG. 1), it is suggested that the decrease of PDV levels during leaf development in turn decreases the rate of chloroplast division, thereby increasing the size of chloroplasts, Supporting this conclusion, the number of chloroplasts in young emerging leaves is similar in the wild type and the PDV1 and PDV2 over-expressers (FIG. 9).

### Example 3

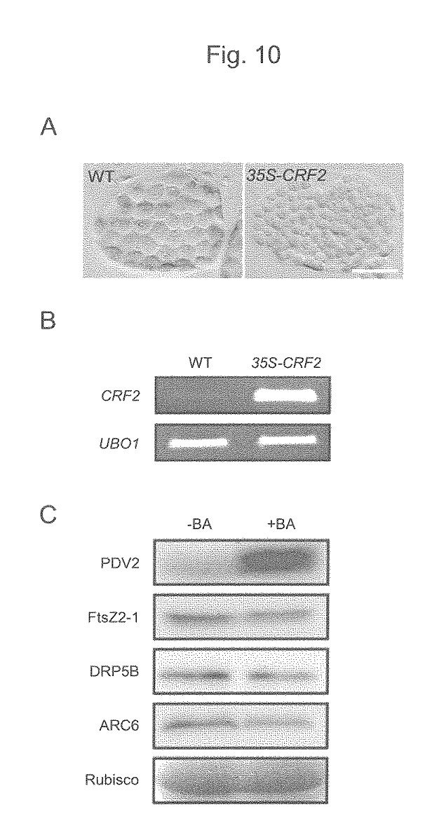

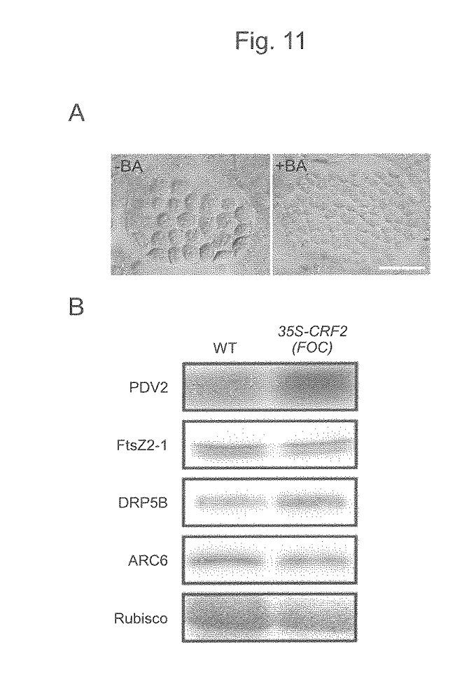

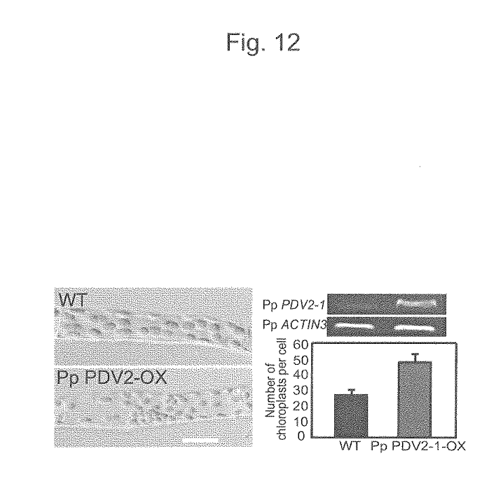

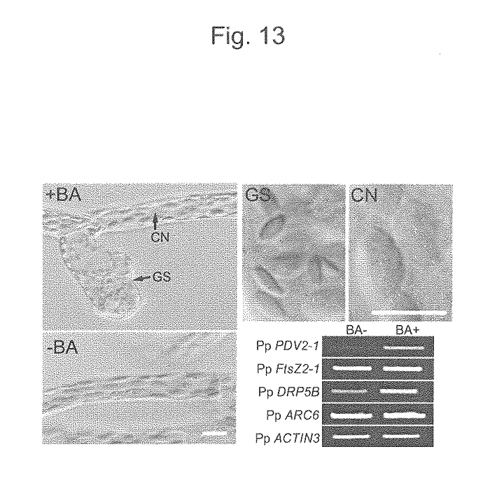

Another FOX line, in which chloroplast division is accelerated, turned out to be an over-expresser of CRF2 (Rashotte et al., 2006). Transgenic plants over-expressing CRF2 as the result of a newly constructed 35S-CRF2 transgene displayed a similar phenotype (FIGS. 10A and 10B), indicating that the phenotype is indeed caused by CRF2 over-expression. Immunoblot analyses showed that the PDV2 level, but not the level of FtsZ, DRP5B or ARC6, was increased in the CRF2 over-expresser (FIG. 10C), suggesting that the increase of the PDV2 level is the cause of the acceleration of chloroplast division in the CRF2 over-expresser.

In land plants, cytokinin regulates numerous growth and developmental processes, including cell division, shoot initiation, and apical meristem function (Kieber, 2002; Kakimoto, 2003). Therefore, the above results suggest that the PDV2 level is regulated by a developmental program responsive to cytokinin. To examine the relationship between PDV levels and cytokinin, wild-type seeds were germinated on medium containing cytokinin (6-benzyladenine: BA). The number of chloroplasts and the PDV2 level between plants treated or untreated with cytokinin were compared. Chloroplasts in the cotyledon cells of cytokinin-treated plants were more numerous than those grown without cytokinin (FIG. 11A). The same pattern of difference was observed when detached true leaves were put on the medium with or without cytokinin. Immunoblot analyses of total plantlets showed that the PDV2 level, but not the level of FtsZ, DRP5B or ARC6, was increased in plants germinated on the cytokinin-containing medium (FIG. 11B). These results suggest that cytokinin, or a developmental program induced by cytokinin, up-regulates PDV2, at least in part through an up-regulation of CRF2, which leads to an increase in the chloroplast division rate.

### Example 4

The results obtained in A. thaliana suggest that PDV1 and PDV2 determine the rate of chloroplast division, at least in the angiosperms. Homologs of PDV1 and PDV2 are encoded in the genomes of other lineages, but are only evident in land plants, suggesting that the genes for these proteins were acquired by the ancestral plant during the transition to terrestrial habitats (Miyagishima et al., 2006; Glynn et al., 2008). To obtain evolutionary insights into the function of PDV proteins, PDV proteins were examined for rate-determination of chloroplast division in mosses. The mosses have branched earliest in land plant evolution (Kenrick and Crane, 1997). The moss Physcomitrella patens is used since the draft genome sequence is available and earlier studies showed involvement of FtsZ in chloroplast division by gene disruption in this organism (Strepp et al., 1998).

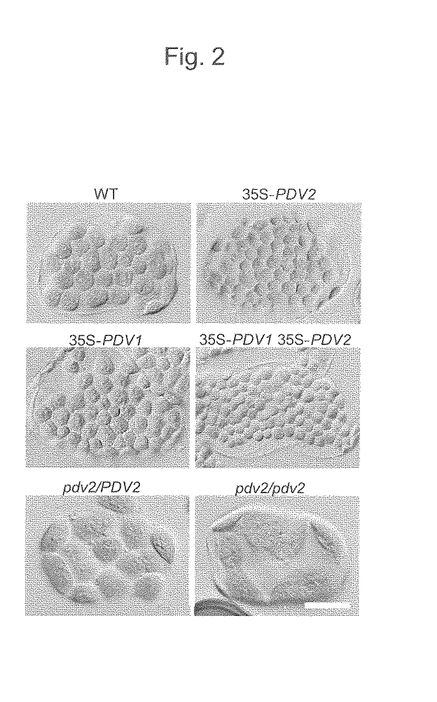

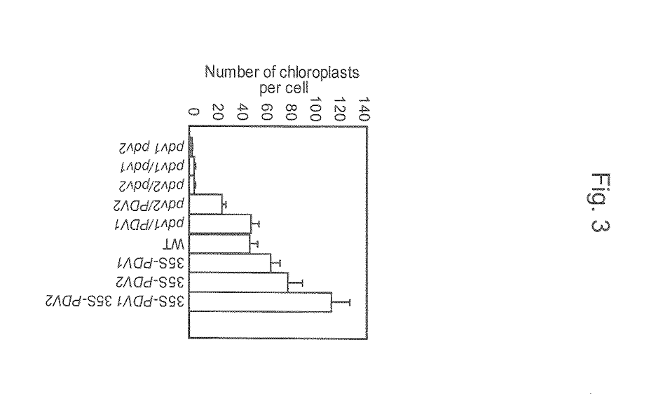

By BLAST and PSI-BLAST (Altschul et al., 1997) searches, four P. patens genes encoding proteins homologous to angiosperm PDV1 and PDV2 were identified. Phylogenic analyses revealed the conservation of PDV1 and PDV2 in angiosperms. By contrast, the amino acid sequence alignment showed that all the moss PDV proteins bear regions found in angiosperm PDV2 but not in angiosperm PDV1; these regions flank both side of the predicted-membrane spanning domain (i.e. both cytosolic and the inter-membrane space). The alignment along with the pattern of the tree topology, suggest that all the moss proteins are orthologous to angiosperm PDV2. Given the recent report that recruitment of PDV1 but not PDV2 is mediated by PARC6 and that PARC6 is unique to vascular plants (Glynn et al., 2009), it is suggested that PDV1, PARC6 and their relationship arose by gene duplications and diversification in ancestral vascular plants after mosses branched out. When the Pp PDV2-1 gene was over-expressed by an E7113 promoter (consisting of seven 35S promoters and additional enhancers; Mitsuhara et al., 1996)-PDV2-1 cDNA transgene, the protonemal cells contained a greater number of smaller sized chloroplasts than did the wild type (FIG. 12), as did the A. thaliana PDV2 over-expresser (FIG. 2).

In contrast to A. thaliana (FIG. 11A), exogenous cytokinin treatment had no effect on the number of protonemal chloroplasts. However, the treatment induced the formation of buds from which gametophytes arise, as reported previously (Reski and Abel, 1985) (FIG. 13). The gametophore shoot apical meristem contained chloroplasts more numerous (per volume) and smaller than those in caulonemal cells (FIG. 13). These results suggest that rate of chloroplast division is increased during the course of bud induction by cytokinin. RT-PCR analyses of the chloroplast division genes showed that the Pp PDV2-1 level was increased by cytokinin treatment (FIG. 13).By contrast, the DRP5B level was slightly up-regulated and the levels of FtsZ2-1, and ARC6 were not changed by the treatment (FIG. 13). The cytokinin treatment up-regulated Pp PDV2-2, Pp PDV2-3, and Pp PDV2-4 but did not change levels of all of the five FtsZ genes (Martin et al., 2009) encoded in the P. patens genome. These results suggest that the PDV2 level changes to modulate the rate of chloroplast division during bud formation in the moss P. patens, and PDV protein function serving as a rate-limiting component of chloroplast division machinery is conserved in land plants.

### REFERENCES

The following references, to the extent that they provide exemplary procedural or other details supplementary to those set forth herein, are specifically incorporated herein by reference. Altschul, S. F., Madden, T. L., Schaffer, A. A., Zhang, J., Zhang, Z., Miller, W., and Lipman, D. J. (1997), Nucleic Acids Res. 25, 3389-3402; Boffey, S. A., and Lloyd, D. (1988) Division and segregation of organelles. (Cambridge: Cambridge University Press); Clough, S. J., and Bent, A. F. (1998), Plant J. 16, 735-743; Colletti, K. S., Tattersall, E. A., Pyke, K. A., Froelich, J. E., Stokes, K. D., and Osteryoung, K. W. (2000), Curr. Biol. 10, 507-516; Gao, H., Kadirjan-Kalbach, D., Froehlich, J. E., and Osteryoung, K. W. (2003), Proc. Natl. Acad. Sci. USA. 100, 4328-4333; Glynn, J. M., Froehlich, J. E., and Osteryoung, K. W. (2008), Plant. Cell 20, 2460-2470; Glynn, J. M., Yang, Y., Vitha, S., Schmitz, A. J., Hemmes, M., Miyagishima, S., and Osteryoung, K. W. (2009), Plant J., in press, DOI: 10.1111/j.1365-313X.2009.03905.x; Gould, S. B., Waller, R. F., and McFadden, G. I. (2008), Annu. Rev. Plant Biol. 59, 491-517; Ichikawa, T., Nakazawa, M., Kawashima, M., Iizumi, H., Kuroda, H., Kondou, Y., Tsuhara, Y., Suzuki, K., Ishikawa, A., Seki, M., Fujita, M., Motohashi, R., Nagata, N., Takagi, T., Shinozaki, K., and Matsui, M. (2006). Plant J. 48, 974-985; Itoh, R., Fujiwara, M., Nagata, N., and Yoshida, S. (2001), Plant Physiol. 127, 1644-1655; Jacqmard, A., Detry, N., Dewitte, W., Van Onckelen, and Bernier, G. (2002). Planta 214, 970-973; Jefferson, R. A., Kavanagh, T. A., and Bevan, M. W. (1987), EMBO J. 6, 3901-3907; Kakimoto, T. (2003), Annu. Rev. Plant Biol. 54, 605-627; Kenrick, P., and Crane, P. R. (1997), Nature 389, 33-39; Kieber, J. J. (2002). Cytokinins: Mar. 27, 2002. The Arabidopsis Book. Rockville, Md.: American Society of Plant Biologists. doi: 10.1199/tab.0063, http://www.aspb.org/publications/arabidopsis/; Kiessling, J., Kruse, S., Rensing, S. A., Harter, K., Decker, E. L., and Reski, R. (2000), J. Cell Biol. 151, 945-950; Kuroiwa, H., Mori, T., Takahara, M., Miyagishima, S., and Kuroiwa, T. (2002), Planta 215, 185-190; Kuroiwa, T., Kuroiwa, H., Sakai, A., Takahashi, H., Toda, K., and Itoh, R. (1998), Rev. Cytol. 181, 1-41; Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., and Higgins, D. G. (2007), Bioinformatics 23, 2947-2948; Lopez-Juez, E., and Pyke, K. A. (2005), Int. J. Dev. Biol. 49, 557-577; Maple, J., and Moller, S. G. (2007), Ann Bot (Lond) 99, 565-579; Maple, J., Vojta, L., Soll, J., and Moller, S. G. (2007), EMBO Rep. 8, 293-299; Martin, A., Lang, D., Heckmann, J., Zimmer, A. D., Vervliet-Seheebaum, M., and Reski, R. (2009), Plant Biol., in press, DOI: 10.1111/j.1438-8677.2008.00174; Mitsuhara, I., Ugaki, M., Hirochika, H., Ohshima, M., Murakami, T., Gotoh, Y., Katayose, Y., Nakamura, S., Honkura, R., Nishimiya, S., Ueno, K., Mochizuki, A., Tanimoto, H., Tsugawa, H., Otsuki, Y., and Ohashi, Y. (1996), Plant Cell Physiol. 37, 49-59; Miyagishima, S. (2005), J. Plant Res. 118, 295-306; Miyagishima, S., Takahara, M., and Kuroiwa, T. (2001), Plant Cell 13, 707-721; Miyagishima, S., Froehlich, J. E., and Osteryoung, K. W. (2006), Plant Cell 18, 2517-2530; Miyagishima, S., Kuwayama, H., Urushihara, H., and Nakanishi, H. (2008), Proc. Natl. Acad. Sci. USA. 105, 15202-15207; Miyagishima, S., Nishida, K., Mori, T., Matsuzaki, M., Higashiyama, T., Kuroiwa, H., and Kuroiwa, T. (2003), Plant Cell 15, 655-665; Mori, T., Kuroiwa, H., Takahara, M., Miyagishima, S., and Kuroiwa, T. (2001), Plant Cell Physiol. 42, 555-559; Nakanishi, H., Suzuki, K., Kabeya, Y., and Miyagishima, S. (2009), Curr. Biol. 19, 151-156; Nishiyama, T., Hiwatashi, Y., Sakakibara, I., Kato, M., and Hasebe, M. (2000), DNA Res. 7, 9-17; Osteryoung, K. W., and Vierling, E. (1995), Nature 376, 473-474; Osteryoung, K. W., Stokes, K. D., Rutherford, S. M., Percival, A. L., and Lee, W. Y. (1998), Plant Cell 10, 1991-2004; Possingham, J. V., and Lawrence, M. E. (1983). Controls to plastid division. Int. Rev. Cytol. 84, 1-56; Pyke, K. A. (1999), Plant Cell 11, 549-556; Rashotte, A. M., Mason, M. G., Hutchison, C. E., Ferreira, F. J., Schaller, G. E., and Kieber, J. J. (2006), Proc. Natl. Acad. Sci. USA. 103, 11081-11085; Raynaud, C., Cassier-Chauvat, C., Perennes, C., and Bergounioux, C. (2004), Plant Cell 16, 1801-1811; Reski, R., and Abel, W. O. (1985), Planta 165, 354-358; Reski, R., Wehe, M., Hadeler, B., Marienfeld, J. R., and Abel, W. O. (1991), J. Plant Physiol. 138, 236-243; Reutter, K., Atzorn, R., Hadeler, B., Schmülling, T., and Reski, R. (1998), Planta 206, 196-203; Reyes-Prieto, A., Weber, A. P., and Bhattacharya, D. (2007), Annu Rev Genet 41, 147-168; Ronquist, F., and Huelsenbeck, J. P. (2003), Bioinformatics 19, 1572-1574; Shimada, H., Koizumi, M., Kuroki, K., Mochizuki, M., Fujimoto, H., Ohta, H., Masuda, T., and Takamiya, K. (2004), Plant Cell Physiol. 45, 960-967; Stamatakis, A. (2006), Bioinformatics 22, 2688-2690; Stokes, K. D., McAndrew, R. S., Figueroa, R., Vitha, S., and Osteryoung, K. W. (2000), Plant Physiol. 124, 1668-1677; Strepp, R., Scholz, S., Kruse, S., Speth, V., and Reski, R. (1998), Proc. Natl. Acad. Sci. USA. 95, 4368-4373; Suzuki, K., Nakanishi, H., Bower, S., Yoder, D. W., Osteryoung, K. W., and Miyagishima, S. (2009), BMC Plant Biol. 9, 38; Vitha, S., McAndrew, R. S., and Osteryoung, K. W. (2001), J. Cell Biol. 153, 111-120; Vitha, S., Froehlich, J. E., Koksharova, O., Pyke, K. A., van Erp, H., and Osteryoung, K. W. (2003), Plant Cell 15, 1918-1933; Yang, Y., Glynn, J. M., Olson, B. J., Schmitz, A. J., and Osteryoung, K. W. (2008), Curr. Opin. Plant Biol. 11, 577-584; Yoon, H. S., Hackett, J. D., and Bhattacharya, D. (2002), Proc., Natl. Acad. Sci. USA. 99, 11724-11729; Yoshida, Y., Kuroiwa, H., Misumi, O., Nishida, K., Yagisawa, F., Fujiwara, T., Nanamiya, H., Kawamura, F., and Kuroiwa, T. (2006), Science 313, 1435-1438.

What is claimed is:

1. A transformed plant with increased number of chloroplasts per cell, comprising an over-expressed foreign DNA which codes for a land-plant specific component protein of a division apparatus PDV1 and/or PDV2, a homologue thereof, or a mutant thereof.

2. The transformed plant according to claim 1, wherein
PDV1 comprises an amino acid sequence as shown in SEQ ID NO: 1,
PDV1 homologue comprises an amino acid sequence having at least 20% identity to the amino acid sequence of SEQ ID NO: 1, and
PDV1 mutant comprises an amino acid sequence of SEQ ID NO: 1 having deletion, addition and/or substitution of one or more amino acids and an activity of increasing the number of chloroplasts per cell when compared with wild type.

3. The transformed plant according to claim 1, wherein
PDV2 comprises an amino acid sequence as shown in SEQ ID NO: 2,
PDV2 homologue comprises an amino acid sequence having at least 20% identity to the amino acid sequence of SEQ ID NO: 2, and
PDV2 mutant comprises an amino acid sequence of SEQ ID NO: 2 having deletion, addition and/or substitution of one or more amino acids and an activity of increasing the number of chloroplasts per cell when compared with wild type.

4. A transformed plant with increased number of chloroplasts per cell, comprising an over-expressed foreign DNA which codes for a transcription factor CRF2, a homologue thereof, or a mutant thereof.

5. The transformed plant according to claim 4, wherein
CRF2 comprises an amino acid sequence as shown in SEQ ID NO: 3,
CRF2 homologue comprises an amino acid sequence having at least 20% identity to the amino acid sequence of SEQ ID NO: 3, and
CRF2 mutant comprises an amino acid sequence of SEQ ID NO: 3 having deletion, addition and/or substitution of one or more amino acids and an activity of increasing the number of chloroplasts per cell when compared with wild type.

6. The transformed plant according to claim 1, wherein the DNA comprises:
(a) a nucleotide sequence as shown in SEQ ID NO: 4 or 5,
(b) a nucleotide sequence having an at least 20% identity to the nucleotide sequence of SEQ ID NO: 4 or 5,
(c) a nucleotide sequence encoding the PDV1 or PDV2 as defined in claim 2 or 3, respectively, or
(d) a nucleotide sequence capable of hybridizing with the nucleotide sequence complement to the nucleotide sequence of SEQ ID NO: 4 or 5 under strength conditions, wherein the nucleotide sequence (a), (b) or (c) codes for a protein having an activity of increasing the number of chloroplasts per cell when compared with wild type.

7. The transformed plant according to claim 4, wherein the DNA comprises:
(a) a nucleotide sequence as shown in SEQ ID NO: 6,
(b) a nucleotide sequence having an at least 20% identity to the nucleotide sequence as shown in SEQ ID NO: 6,
(c) a nucleotide sequence encoding CRF2 as defined in claim 5,
(d) a nucleotide sequence capable of hybridizing with the nucleotide sequence complement to the nucleotide sequence of SEQ ID NO: 6 under strength conditions, wherein the nucleotide sequence (a), (b) or (c) codes for a protein having an activity of increasing the number of chloroplasts per cell when compared with wild type.

8. Progeny of the transformed plant according to claim 1 or 4.

9. A cell, tissue, organ or seed derived from the transformed plant according to any one of claims 1 to 7 or the progeny according to claim 8.

10. A method for producing a transformed plant according to claim 1, comprising the following steps of:
(1) introducing a vector comprising the DNA as defined in claim 1 or 4 into cells of plant,
(2) selecting a transformed cell over-expressing the DNA, from the transformed cells or step (1), and
(3) generating the transformed plant from the transformed cells of step (2).


SEQUENCE LISTING (65 sequences)

SEQ ID NO: 1 - protein, 272 aa, Arabidopsis thaliana
MGEMEIEEIEAVLEKIWDLHDKLSDEIHLISKSHFLKSVKPSNRSEKRKNPHGNSGEDKRPGYVFIKGFAVDDNDSTIQEAKSLNAIRTALENLEDQLEFFHTIHTQQRTEKDVAIARLEQSRILLAMRLAEHHGKNYGVLEEALAFVGSIKSNSHYVSPDHLYDSSRNPDGANSIPDGIESNFVINAFASTFGFAKRALGFNHVKGVLGNAAIFAISVVAMLHLHQVATSEHHLQKKEDRFYRSQQRKTYGRDKSSADRSLDHLDVMMARG

SEQ ID NO: 2 - protein, 307 aa, Arabidopsis thaliana
MEDEEGIGLILARATELRLKISDCIDNSSTTVSDNGDGNEDLSPGEGRKSEIIGNQDKDFDSISSEDVDEAEAERLLRIRDALEALESQLASLQNLRQRQQYEKQLALSEIDYSRKMLLEKLKEYKGKDFEVLRETTTFAGERVDYENDLLLPPYPVHPPLSLGLDNNNGYLSHLPSKKKSDANGFGSGHVRNEAEAKSPNGGSGGSSHGVIRFLGSVAKIVLPIIGVISLLSASGYGPEMRKRGASLNLFGLLPHRATRGKRTPNQCPPGKVLVIEDGEARCLVKERVEIPFDSVVAKRDVTYGYG

SEQ ID NO: 3 - protein, 343 aa, Arabidopsis thaliana
MEAEKKMVLPRIKFTEHKTNTTTIVSELTNTHQTRILRISVTDPDATDSSSDDEEEEHQRFVSKRRRVKKFVNEVYLDSGAVVTGSCGQMESKKRQKRAVKSESTVSPVVSATTTTTGEKKFRGVRQRPWGKWAAEIRDPLKRVRLWLGTYNTAEEAAMVYDNAAIQLRGPDALTNFSVTPTTATEKKAPPPSPVKKKKKKNNKSKKSVTASSSISRSSSNDCLCSPVSVLRSPFAVDEFSGISSSPVAAVVVKEEPSMTTVSETFSDFSAPLFSDDDVFDFRSSVVPDYLGGDLFGEDLFTADMCTDMNFGFDFGSGLSSWHMEDHFQDIGDLFGSDPLLAV

SEQ ID NO: 4 - DNA, 819 bp, Arabidopsis thaliana
atgggagaaatggagatcgaagaaatcgaagctgttcttgagaaaatctgggatctacatgacaagcttagcgatgagattcacttgatttcgaagtctcactttctcaaatcggttaaaccttcaaatagatcggagaagagaaagaatcctcacggaaattccggcgaagataagcgacctggttatgttttcatcaaaggattcgccgtcgatgataacgactccacgattcaggaggcgaagagtcttaacgcaattcgaaccgctcttgagaaccttgaagaccagctcgagtttttccatactatacatacacagcagcgaacagagaaagatgttgcgattgctcggcttgaacagagcaggatattactagcaatgagattagctgaacaccacggcaagaactatggagtcctagaggaagctcttgcatttgttgggagcattaaatccaactcgcattacgtttcacctgatcacctctacgattcctctcgaaacccagacggagcaaactctattccagatggaatagagtcaaactttgtaatcaacgcctttgcctcgactttcgggtttgccaagagagcacttggattcaaccatgtgaaaggcgtacttggaaacgctgctatatttgccataagcgtggtcgctatgctacatcttcaccaagtggctactagtgaacatcatctgcagaagaaagaagacagattctacagaagccaacaaaggaaaacatacggaagagataagtcttcagctgacagaagtttggatcacttggacgtaatgatggctcgtggttaa

SEQ ID NO: 5 - DNA, 924 bp, Arabidopsis thaliana
atggaagacgaagaaggcatcgggttaattttagccagagccaccgagctcagactcaagatcagcgattgtatcgataactccagtaccaccgtcagcgacaacggcgatggaaacgaggatctttctcccggagaaggaagaaaaagcgagattattgggaatcaagacaaggattttgactcaatcagctctgaggatgtagatgaagcagaagctgaacggcttttgcgtattcgtgatgctcttgaagctcttgagtctcagcttgcttctttacagaatctaaggcaaagacaacaatatgagaagcaactggctctctctgagattgattacagtaggaaaatgttgcttgagaagcttaaggagtacaaagggaaagattttgaagtgttacgtgagactacgacttttgctggtgaaagagttgattatgagaatgatcttttacttcctccttatcctgtccatcctcctctctcccttggtttagataataataatggttacctatctcacttaccttcgaagaagaaatcggatgcgaatgggttcggctcaggacatgtgaggaacgaggcagaggcgaaaagtcccaacggaggatctggaggttcgagtcatggggttattcgcttcttaggctctgtcgcaaagatcgtgcttccaatcatcggtgttatctctctattgtctgcatcaggatatggtccagagatgaggaaaagaggcgcttccttgaacctttttggacttctcccgcaccgagctaccagagggaagagaacacctaaccaatgtccaccagggaaagttcttgtgattgaagatggagaagcacggtgccttgttaaggaaagagtcgaaataccatttgactctgttgtagcgaaacgcgatgtaacttacggatacggttga

SEQ ID NO: 6 - DNA, 1032 bp, Arabidopsis thaliana
atggaagcggagaagaaaatggttctaccgagaatcaaattcacagagcacaaaaccaacacgacaacaatcgtatcggagttaaccaacactcaccaaaccaggattcttcgtatctcagtcactgacccagacgctactgattcctccagtgacgacgaagaagaagaacatcaacgctttgtctctaaacgccgtcgtgttaagaagtttgtcaacgaagtctatctcgattccggtgctgttgttactggtagttgtggtcaaatggagtcgaagaagagacaaaagagagcggttaaatcggagtctactgtttctccggttgtttcagcgacgacgactacgacgggagagaagaagttccgaggagtgagacagcgtccatggggaaaatgggcggcggagataagagatccgttgaaacgtgtacggctctggttaggtacttacaacacggcggaagaagctgctatggtttacgataacgccgctattcagcttcgtggtcccgacgctctgactaatttctcagtcactccgacaacagcgacggagaagaaagccccaccaccgtctccggtgaagaagaagaagaagaaaaacaacaaaagcaaaaaatccgttactgcttcttcctccatcagcagaagcagcagcaacgattgtctctgctctccggtgtctgttctccgatctcctttcgccgtcgacgaattctccggcatttcttcatcaccagtcgcggccgttgtagtcaaggaagagccatccatgacaacggtatctgaaactttctctgatttctcggcgcccttgttctcagatgatgacgtgttcgatttccggagctcagtggttcccgactatctcggcggcgatttatttggggaagatctattcacggcggatatgtgtacggatatgaacttcggattcgatttcggatccggattatccagctggcacatggaggaccattttcaagatatcggggatctattcgggtcggatcctcttttagctgtttaa

SEQ ID NO: 7 - DNA, 30 bp, Arabidopsis thaliana
gtacgtatttttacaacaattaccaacaac

SEQ ID NO: 8 - DNA, 30 bp, Arabidopsis thaliana
ggattcaatcttaagaaactttattgccaa

SEQ ID NO: 9 - DNA, 13 bp, Arabidopsis thaliana
ccccccccccccd

SEQ ID NO: 10 - DNA, 30 bp, Arabidopsis thaliana
agtgctagcacaattaaagtgagaattttc

SEQ ID NO: 11 - DNA, 30 bp, Arabidopsis thaliana
agtcccgggctggggtgcgaatgggctgat

SEQ ID NO: 12 - DNA, 30 bp, Arabidopsis thaliana
atgtctagaaaacactccaaattacttgtc

SEQ ID NO: 13 - DNA, 30 bp, Arabidopsis thaliana
catcccgggatgtcaggcaatgaacattac

SEQ ID NO: 14 - DNA, 30 bp, Arabidopsis thaliana
gcttctagatcctcttcttatctctctctc

SEQ ID NO: 15 - DNA, 30 bp, Arabidopsis thaliana
gtacccgggttaaacagctaaaagaggatc

SEQ ID NO: 16 - DNA, 21 bp, Arabidopsis thaliana
gcttagaaactccgacaagaa

SEQ ID NO: 17 - DNA, 30 bp, Arabidopsis thaliana
ggtaccccgggatccaattaacccgatgcc

SEQ ID NO: 18 - DNA, 30 bp, Arabidopsis thaliana
ggatcccggggtaccatggaagacgaagaa

SEQ ID NO: 19 - DNA, 21 bp, Arabidopsis thaliana
accaagtggtataagttggga

SEQ ID NO: 20 - DNA, 27 bp, Arabidopsis thaliana
ggatccatggtgagcaagggcgaggag

SEQ ID NO: 21 - DNA, 24 bp, Arabidopsis thaliana
tttggtacctcctcctcccttgta

SEQ ID NO: 22 - DNA, 30 bp, Arabidopsis thaliana
ttcatggtaattattctatcactattcaag

SEQ ID NO: 23 - DNA, 30 bp, Arabidopsis thaliana
ggtacctgatgcaagaacagagccttcagt

SEQ ID NO: 24 - DNA, 39 bp, Arabidopsis thaliana
ggggtaccggaggaggaggaatggtgagcaagggcgagg

SEQ ID NO: 25 - DNA, 32 bp, Arabidopsis thaliana
tccccgcggttacttgtacagctcgtccatgc

SEQ ID NO: 26 - DNA, 30 bp, Arabidopsis thaliana
aaaggatccgcttagaaactccgacaagaa

SEQ ID NO: 27 - DNA, 30 bp, Arabidopsis thaliana
tttggatccaattaacccgatgccttcttc

SEQ ID NO: 28 - DNA, 30 bp, Arabidopsis thaliana
atgtctagagacgcactgtcactgctacgg

SEQ ID NO: 29 - DNA, 30 bp, Arabidopsis thaliana
atgtctagaagtaaaacacggtgaaacgta

SEQ ID NO: 30 - DNA, 30 bp, Arabidopsis thaliana
atctctagattcatgttttttcccctggtg

SEQ ID NO: 31 - DNA, 30 bp, Arabidopsis thaliana
tactctagacgtcaccgattttgctgatac

SEQ ID NO: 32 - DNA, 25 bp, Arabidopsis thaliana
caccatggaagacgaagaaggcatc

SEQ ID NO: 33 - DNA, 21 bp, Arabidopsis thaliana
tcaaccgtatccgtaagttac

SEQ ID NO: 34 - DNA, 30 bp, Physcomitrella patens
atgcccgggattaggtgtgggggtaggatc

SEQ ID NO: 35 - DNA, 30 bp, Physcomitrella patens
atgcccgggtcagccgcggccatgcaggac

SEQ ID NO: 36 - DNA, 20 bp, Arabidopsis thaliana
accgctcttgagaaccttga

SEQ ID NO: 37 - DNA, 20 bp, Arabidopsis thaliana
gaggcaaaggcgttgattac

SEQ ID NO: 38 - DNA, 26 bp, Arabidopsis thaliana
gtagtcaaggaagagccatccatgac

SEQ ID NO: 39 - DNA, 26 bp, Arabidopsis thaliana
catgtgccagctggataatccggatc

SEQ ID NO: 40 - DNA, 20 bp, Arabidopsis thaliana
ggccaagatccaagacaaag

SEQ ID NO: 41 - DNA, 20 bp, Arabidopsis thaliana
gttgacagctcttgggtgaa

SEQ ID NO: 42 - DNA, 27 bp, Physcomitrella patens
caagtggtggaggaagcgatgacattc

SEQ ID NO: 43 - DNA, 25 bp, Physcomitrella patens
gtgcgtcgttgtgctcatatcagca

SEQ ID NO: 44 - DNA, 22 bp, Physcomitrella patens
atcgagctgacaaaatgctaac

SEQ ID NO: 45 - DNA, 24 bp, Physcomitrella patens
gtaataagacaggctttactgttg

SEQ ID NO: 46 - DNA, 21 bp, Physcomitrella patens
cagtgaacacacaaccatcta

SEQ ID NO: 47 - DNA, 21 bp, Physcomitrella patens
ttgtgttcagtgaatgctagg

SEQ ID NO: 48 - DNA, 21 bp, Physcomitrella patens
gaaagcagaagagttctgttg

SEQ ID NO: 49 - DNA, 22 bp, Physcomitrella patens
cactaacaatacttttcctgtg

SEQ ID NO: 50 - DNA, 21 bp, Physcomitrella patens
agatgttagagctgtaatgag

SEQ ID NO: 51 - DNA, 22 bp, Physcomitrella patens
tggcaataatcgttacatggat

SEQ ID NO: 52 - DNA, 21 bp, Physcomitrella patens
tgctgaagaagctgcaattca

SEQ ID NO: 53 - DNA, 22 bp, Physcomitrella patens
caagttgagagttgattgcttc

SEQ ID NO: 54 - DNA, 25 bp, Physcomitrella patens
gaggtaaatgctgcagcagaggtga

SEQ ID NO: 55 - DNA, 25 bp, Physcomitrella patens
tggtatattgatcgcagagccattg

SEQ ID NO: 56 - DNA, 22 bp, Physcomitrella patens
cagcattgagtgccattcagtc

SEQ ID NO: 57 - DNA, 22 bp, Physcomitrella patens
gcttaataatgatgagcaatcc

SEQ ID NO: 58 - DNA, 21 bp, Physcomitrella patens
gtgttcgaactgtagacgatg

SEQ ID NO: 59 - DNA, 21 bp, Physcomitrella patens
gctttcagagtatcgattgag

SEQ ID NO: 60 - DNA, 26 bp, Physcomitrella patens
atgttgctcaggttcgtagagcattg

SEQ ID NO: 61 - DNA, 26 bp, Physcomitrella patens
tagagggaacatcatggtgttccagg

SEQ ID NO: 62 - DNA, 26 bp, Physcomitrella patens
cagccatatcatgggtaggaccaagt

SEQ ID NO: 63 - DNA, 26 bp, Physcomitrella patens
caagaattcagtcgatgacggctctg

SEQ ID NO: 64 - DNA, 24 bp, Physcomitrella patens
cggagaggaagtacagtgtgtgga

SEQ ID NO: 65 - DNA, 24 bp, Physcomitrella patens
accagccgttagaattgagcccag